(19) United States
(12) Patent Application Publication
(10) Pub. No.: US 2019/0279182 A1
(43) Pub. Date: Sep. 12, 2019

(54) PARTICIPATIVE HEALTH KIOSK

(71) Applicant: Steelcase Inc., Grand Rapids, MI (US)

(72) Inventors: Alan Rheault, Grand Rapids, MI (US); DeVon Robert Robinson, Grand Rapids, MI (US)

(21) Appl. No.: 16/421,205

(22) Filed: May 23, 2019

Related U.S. Application Data
(63) Continuation of application No. 14/537,183, filed on Nov. 10, 2014.
(60) Provisional application No. 61/908,417, filed on Nov. 25, 2013.

(51) Int. Cl. G06Q 20/18; G16H 10/60; G05B 15/02

(52) U.S. Cl. CPC G06Q 20/18; G05B 15/02; G16H 10/60

(57) ABSTRACT

A participative kiosk configured to provide an interactive and adaptive environment is provided. The kiosk is comprised of a user interface, a user support, at least one biometric sensor, an audio sensor and an optical sensor. The kiosk includes a display and at least one audio speaker. The user support is positioned to provide a user with a view of the user interface. The biometric sensors are configured to measure real-time biometric data associated with the user. The auditory sensor receives vocal input commands from the user and the optical sensor detects movement of the user within the kiosk. The control system is configured to communicate with the user interface, the biometric sensors, the auditory sensor, and the optical sensor. The control system operable also is configured to control at least one environmental condition of the kiosk in response to an input from at least one of the biometric, auditory and optical sensors.

### CROSS REFERENCE TO RELATED APPLICATIONS

This application is a continuation of U.S. application Ser. No. 14/537,183, filed Nov. 10, 2014, which is a non-provisional application of, and claims priority from, and incorporates the disclosure of U.S. Provisional Patent Application No. 61/908,417, filed Nov. 25, 2013.

### TECHNICAL FIELD

The present invention relates to a health kiosk, and more particularly, to a health kiosk that provides interactive and adaptive response to biometric, user and third party inputs.

### BACKGROUND OF THE INVENTION

Kiosks for providing limited on-site triage are well known. With the expansion of the internet, it has become possible to receive limited health data about a kiosk user based on the user's health records or on results from the on-site triage or testing. However, known kiosks have generally been either loosely or completely unconnected to electronic medical records and contextual epidemiological data. The present invention is provided to provide advantages and aspects not provided by prior health kiosks. A full discussion of the features of the present invention is deferred to the following detailed description, which proceeds with reference to the accompanying drawings.

### SUMMARY OF THE INVENTION

According to the present invention, a participative kiosk configured to provide an interactive and adaptive environment is provided. The kiosk includes a user interface, a user support, at least one biometric sensor, an audio sensor and an optical sensor. The kiosk includes a display and at least one audio speaker. The user support is positioned to provide a user with a view of the user interface. The biometric sensors are configured to measure real-time biometric data associated with the user. The auditory sensor receives vocal input commands from the user and the optical sensor detects movement of the user within the kiosk. A control system is configured to communicate with the user interface, the biometric sensors, the auditory sensor, and the optical sensor. The control system also is configured to control at least one environmental condition of the kiosk in response to an input from one or more of the biometric, auditory and optical sensors.

A system configured to provide an interactive and adaptive response to biometric inputs and user inputs is also provided. The system has a user kiosk that includes a user interface having a display, at least one audio speaker and a user support positioned to provide a user with a view of the user interface. The kiosk also includes a sensor system configured to generate user health output data. The sensor system has biometric sensors configured to measure real-time biometric data associated with the user, an auditory sensor for receiving vocal input commands from the user, and an optical sensor for detecting movement of the user within the kiosk. A control system is also provided. The control system is configured to adjust at least one environmental condition of the kiosk in response to one or both of user health output data generated by the sensor system and a diagnostic output. A transceiver is coupled to the kiosk. The transceiver is configured to receive and retransmit user health output data, input transmissions from the user interface and diagnostic outputs.

A method for use in a client-server interactive participative health system for providing interactive and adaptive response to biometric and user inputs is also provided. According to one embodiment of the method, a transmission of a unique identifier is received. The unique identifier corresponds to either a single user or a predetermined class of users. The method includes the step of communicating with an electronic health record database associated with the unique identifier. User health output data measured by biometric sensors within a participative health kiosk are received, and a diagnostic output is generated. At least one command signal is generated based on the generated diagnostic output. The generated command signal and signals are then transmitted.

A non-transitory computer readable medium is also provided. The non-transitory computer readable medium is comprised of a plurality of machine-readable instructions for causing a processor to execute a method for providing automated user assessment and care. The method includes the step of receiving a transmission of a unique identifier, wherein the unique identifier corresponds to one of either a single user or a predetermined class of users. The method also includes the step of communicating with an electronic health record database associated with the unique identifier. At least one user health output data is received; the health output data is measured by at least one biometric sensor within a participative health kiosk. A diagnostic output is generated and one of a plurality of command signals based on the generated diagnostic output is transmitted.

The features and advantages of the invention will be apparent from the following specification taken in conjunction with the following drawings.

### BRIEF DESCRIPTION OF THE DRAWINGS

To understand the present invention, it will now be described by way of example, with reference to the accompanying drawings in which:

FIG. 1 is a participative health kiosk system according to the present invention;

FIG. 2 is a flowchart a flow chart illustrating the method for a method for providing interactive and adaptive response to biometric and user inputs according to the present invention;

FIG. 3 is a block diagram of a computer used in connection with the present invention;

FIGS. 4A-4C are various views of a participative health kiosks according to the present invention;

FIGS. 5A-5F illustrate a portion of a self-navigating user interaction with the system interaction with a participative health kiosks according to the present invention; and,

FIGS. 6A-6C illustrate a portion of a directed user interaction with the system interaction with a participative health kiosks according to the present invention.

The components in the drawings are not necessarily to scale, emphasis instead being placed upon clearly illustrating the principles of the present invention.

### DETAILED DESCRIPTION

While this invention is susceptible to embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

The embodiments disclosed herein provide a system and methods for providing an interactive and adaptive response to biometric inputs, user inputs and remote third-party participant's inputs. More particularly, the system is an interactive kiosk system that allows for directed bio-sensing, self-interaction or multi-party adaptive environmental interaction. The system described herein provides adaptive outputs in response to passive sensing and interactive inputs that extend the reach of a physician or clinician, and helps to facilitate transitions between clinical and consultation roles. Furthermore, the system contemplates various health portal interactions. The system can be employed as either a self-navigating, bio-sensing distributed health portal, as a directed social-sensing distributed health portal, or a combination thereof. When employed as a self-navigating, bio-sensing distributed health portal, a user interacts solely with the system. When employed as a directed social-sensing distributed health portal, both the user and a remote participant interact with the system. In such an instance, the remote participant can be, for example, a physician, a clinician, a family member, a health coach or any member of a user's wellness team who cannot be present.

The system and related methods facilitate measurement, analysis, preliminary diagnosis and communication of health-related data associated with a user. The system is able to measure, analyze, record and/or communicate data associated with the user from a variety of input devices including biometric sensing inputs, application program interfaces (e.g., GoogleMaps, Center for Disease Control sick maps, etc.) inputs provided by the user through I/O devices, input by remote third-party participants or a combination of all of these inputs. The system also is configured to adapt the user environment within the kiosk in response to any, or all, of these inputs. It will be understood that the system and method herein is implemented through a combination of computer hardware, sensor technology and medical device hardware. The system also employs a combination of computer and input device software. The embodiments described, however, will generally be referred to herein as kiosk or system.

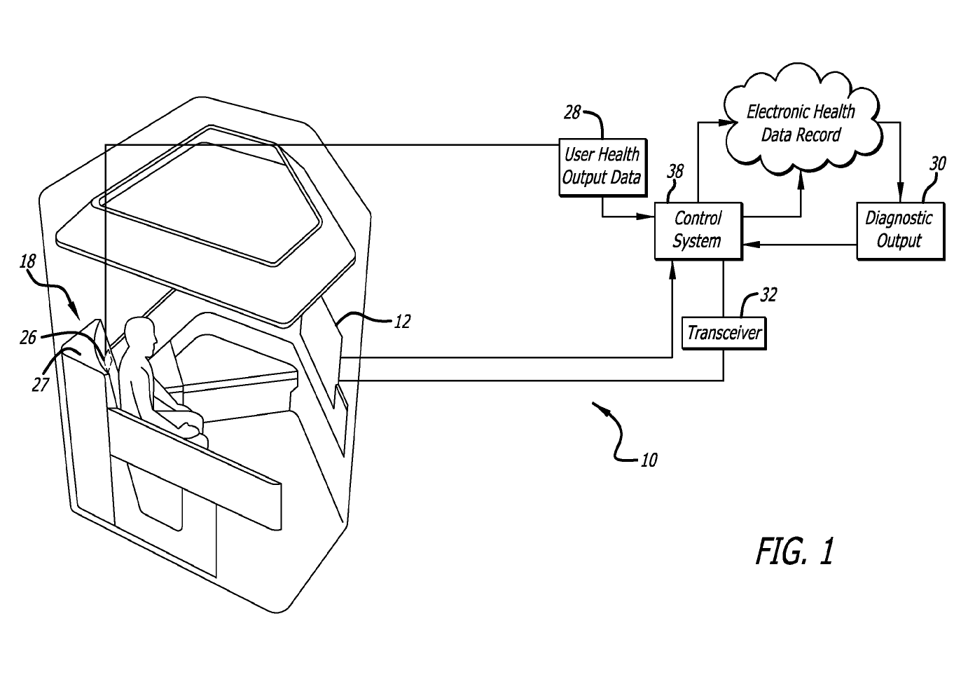

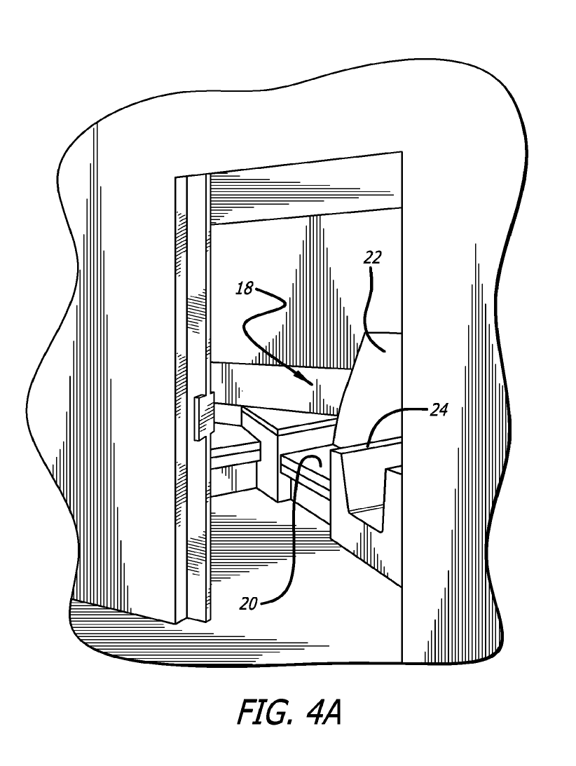

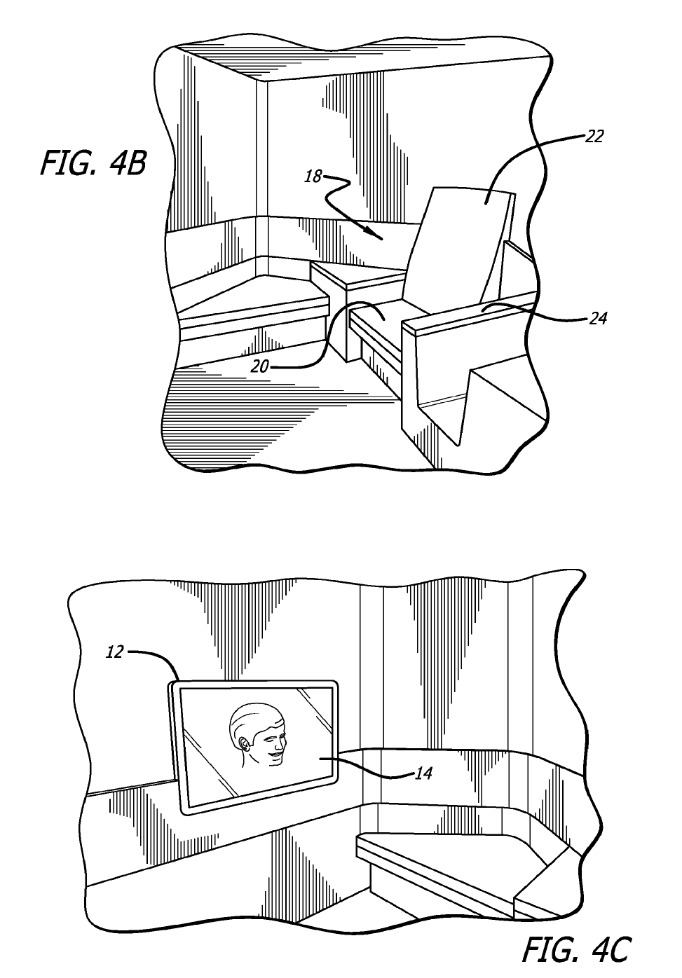

As shown in FIGS. 1 and 4A-4C, the kiosk 10 includes a user interface 12 having a display 14 and at least one audio speaker, and a user support 18. The kiosk 10 is configured such that the user support 18 is fixed or movable to a location within the kiosk 10 that provides a user with a view of the user interface 12. The user support 18 may include a seat portion 20 and, optionally, a back portion 22 and arm supports 18. As the kiosk 10 is meant to provide a soothing interactive environment, it is preferable that the user support 18 be ergonomically designed to maximize comfort and accessibility to the seat portion 20 of the user support 18. The user support 18 may also include heating and cooling mechanisms, all of which are known to those of ordinary skill. In those instances in which the support includes a back portion 22 and/or arm rest, it is preferable that the back portion 22 and arm rest be adjustable to maximize or adjust to the comfort needs of the user. It is contemplated that the kiosk 10 also includes a sanitization system that is triggered upon entry or exit from the kiosk 10. In those instances, the sensor system 26 may receive a signal that a user is entering or exiting the kiosk 10 at which point the sanitization system will be activated to sterilize the kiosk 10.

The kiosk 10 also includes a sensor system 26 configured to generate user health output data 28. User health output data 28 is generally any user health information derived, at least in part, real-time by the sensor system 26, input by a user through the user interface 12 and/or input by a remote participant at the time of use of the system. As will be discussed herein, user health output data 28 is compared against a user's electronic health record or against internal or external epidemiological data during a diagnostic session. It will be understood that the term electronic health record as used herein also can include electronic medical records. A diagnostic output 30 is generated based on one or more of these comparisons. The diagnostic output 30 can be based on a comparison of one or all of user conditions stored in an electronic health data database to the user health output data 28, an input transmission from the user interface 12 and/or input by a remote participant. Alternatively, the generated diagnostic output 30 may be based on a comparison of non-user epidemiological data to the user health output data 28, an input transmission from the user interface 12 and/or an input transmission by a remote participant (or a combination thereof). The non-user epidemiological data can be stored in an epidemiological database or transmitted and retrieved from the Internet or social media feeds. User health output data 28 also may be transmitted to a remote participant in the diagnostic session. It is contemplated that the participant be a non-user human participant (such as, for example, a physician, an employer or a health institution) or a remote computer.

The sensor system 26 can include one or more types of sensors. For example the sensor system 26 may include biometric sensors, auditory sensors configured to receiving vocal input commands from the user, optical sensors for detecting movement within the kiosk 10, or any invasive or non-invasive medical sensors suitable for gathering user health information. According to one embodiment, biometric sensors are configured to measure real-time biometric health information of a user. For example the biometric sensors may be any one, or combination, of a heart rate sensor, a respiratory rate sensor, a blood oxygenation sensor, a blood pressure sensor, a thermometer, a blood glucose sensor, an electrocardiogram sensor, electromyography sensors, pressure resistance sensor to determine muscle strength, weight scale, body mass index sensors, galvanic skin response sensors to determine stress levels, eye tracking sensors, variable heart rate monitors, voice and facial coding sensors and vision testers. The biometric sensors can also include biometric identity sensors such as, for example, fingerprint sensors, retinal sensors or other sensors suitable for recognizing individuals based on unique physical and behavioral traits. It also will be understood that the sensors included in the sensor system 26 may include invasive or non-invasive sensors, or a combination of invasive and non-invasive sensors.

In one embodiment of the present invention, at least some of the biometric sensors are disposed in the user support 18. For example, the user support 18 armrest may include sensors that capture glucose levels, while the seat portion 20 may include sensors that capture blood pressure, respiration, oxygenation, heart rate, weight and other levels. It will be understood, however, that sensors may be disposed in any part of the kiosk 10 best suited for retrieving data from the user. For example, one or two biometric sensors will be preferably disposed at the entry of the kiosk. Biometric sensors also can be attached to or near cameras or infrared sources.

A control system 38 is also provided. The control system 38 is disposed in operable communication with the kiosk 10 and is configured to adjust at least one environmental condition of the kiosk 10. More particularly, the control system 38 will adjust environmental conditions of the kiosk 10 in response to a user health output data 28 generated by the sensor system 26, an input transmission from the user interface 12, a diagnostic output 30 or a combination thereof. For example, the control system 38 may dim the lights in the kiosk 10 if the control system 38 receives a transmission indicative of high user stress levels. In another example, the temperature of the kiosk 10 may be adjusted based on user health output data 28 generated by the sensor system 26 or an input transmission from the user interface 12. In yet another example, all or part of the user support 18 may adjust based on user health data outputs, user input transmissions or remote participant input transmission. In still other examples: the volume is adjusted for a user who is hard of hearing; a burst of air can be emitted to help get the attention of a distracted or tired user; the color of the lighting can change (for example, studies have shown that amber lighting is calming and blue lighting is stimulating); the font size on the screen can be adjusted; or a scent or sound (e.g., of waves, rain or birds singing) can be introduced into the kiosk 10. It will be understood that the control system 38 can be integrated into the kiosk 10 or disposed remotely from the kiosk 10 without departing from the present invention.

According to one embodiment, the control system 38 is operable to execute a plurality of vignettes in response to a user health output data 28 generated by the sensor system 26 or a diagnostic output 30. For example, a user may be instructed to drink fluids in response to a health output data indicating that the user has a heightened temperature reading.

A transceiver 32 is coupled to the kiosk 10. The transceiver 32 is configured to receive and retransmit user health output data 28, input transmissions from the user interface 12 and diagnostic outputs. The transceiver 32 is also configured to receive a transmission of unique identifiers corresponding to either a single user or a class of users. In the instance of correspondence to a single user, the unique identifier, for example, may be a user's social security number, employee identification number, health record number or other identifier that uniquely corresponds to a particular user. In the instance of correspondence to a class of users, the unique identifier, for example, may be a geographic location, a work site, a group of persons identified as at high or low risk for a particular disease or malady or any other identifier that uniquely corresponds to a collective group of users.

The transceiver 32 may transmit and receive communications to and from an electronic health records database or a single electronic health record associated with a unique identifier. The transceiver 32 also may be configured to receive and transmit communications to and from remote databases and remote participants. The transceiver 32 may also transmit prompts to the user interface 12 or transmit data from the kiosk 10. For example, the transceiver 32 is operable to receive and transmit recommendations, analyses, diagnoses and epidemiological data to and from the kiosk 10. In this way the kiosk 10 or the user may interface with remote persons and databases.

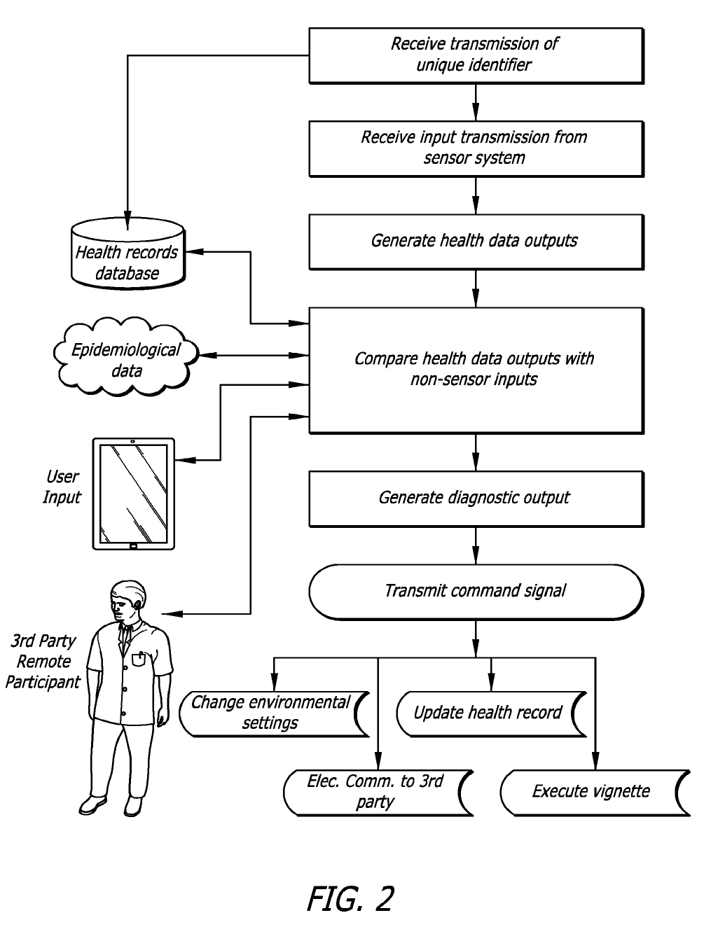

As shown in FIG. 2, a method for use in a client-server interactive participative health system is provided. The method provides interactive and adaptive response to biometric and user inputs. According to the method, a transmission of a unique identifier is received. The unique identifier can correspond to one of either a single user or a predetermined class of users. The unique identifier may be received from an I/O device at or in the kiosk 10. Alternatively, the unique identifier may be received as an output transmission from a biometric sensor (e.g., retinal scan, fingerprint, facial recognition, etc.) or voice input through known voice input devices. In yet another embodiment, the unique identifier may be received as a transmission from a remote third-party server or device.

The system will then communicate with a health record database associated with the unique identifier and user health output data is received. User health output data is measured by biometric sensors within a participative health kiosk. A diagnostic output is then generated and, based on the user health output data, one of a plurality of command signals is transmitted. These command signals may include an environmental command signal for changing an environmental setting of an enclosed environment.

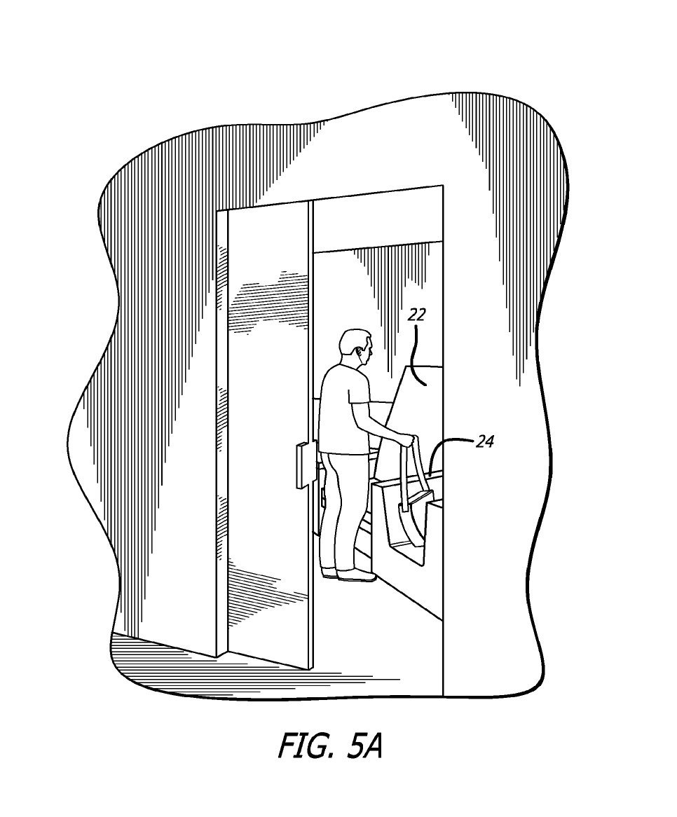

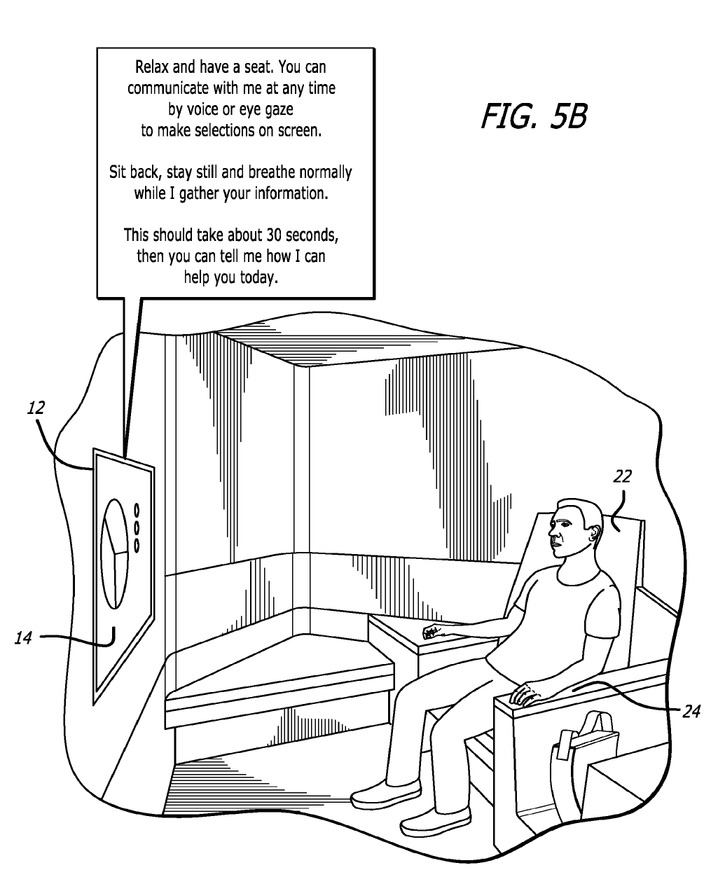

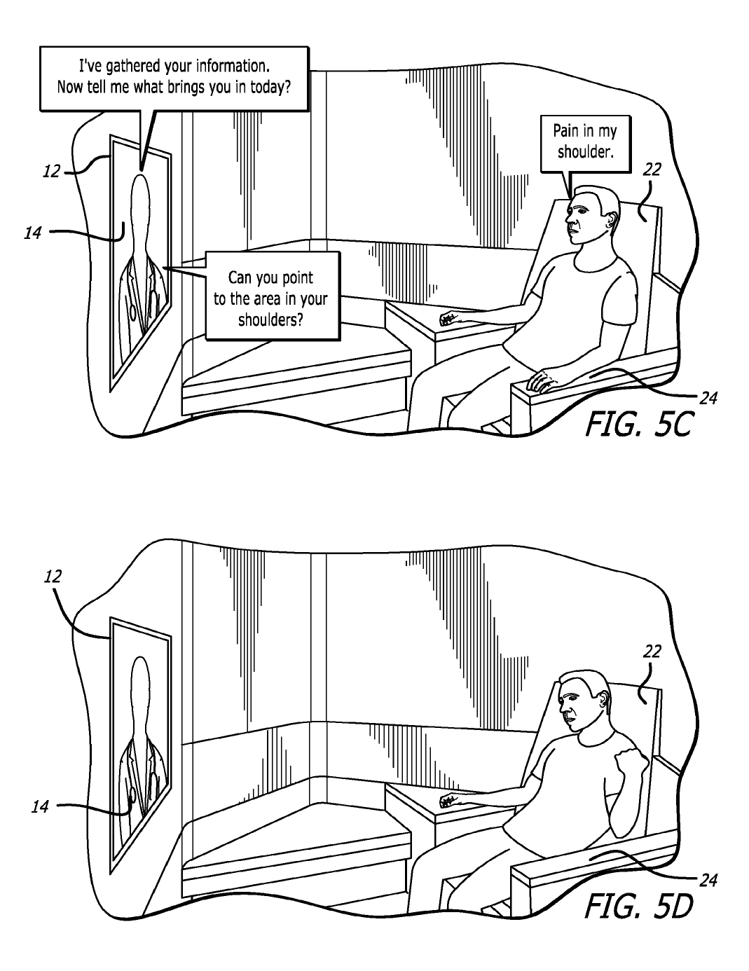

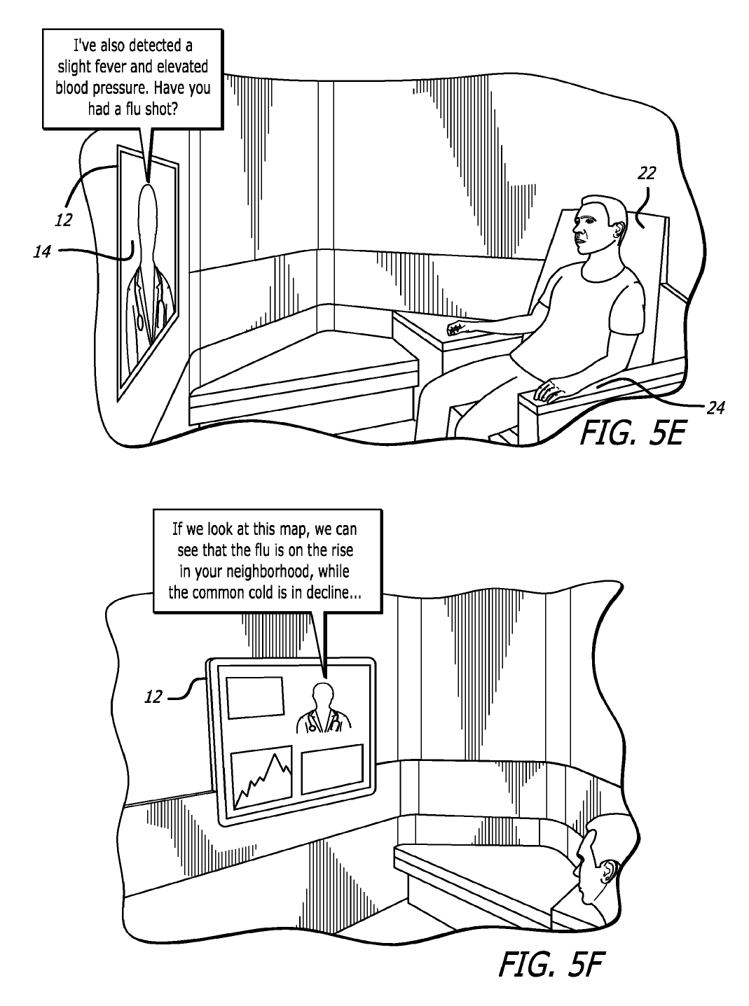

A diagnostic routine can be executed by transmitting at least one prompt to a user interface and receiving at least one input from the user interface. The diagnostic routine may present as an exchange between the user and an electronic respondent (unilateral diagnostic routine). As shown in FIGS. 5A-5F, in a unilateral diagnostic exchange, the user interface 12 may display a string of text prompts and responses. It is contemplated that the text also be provided in audio form. Alternatively, an avatar displayed on the user interface 12 may provide audible and visual prompts and responses. It will also be understood that the entire diagnostic routine may also include visual or sensory cues (e.g., light dimming, temperature adjustment, and white noise) that stimulate prompts and responses. An exemplary diagnostic exchange in a self-navigated, bio-sensing distributed health interaction between a user and an avatar is set forth below:
Avatar: Welcome. Relax and have a seat. You can communicate with me at any time by voice or by making selections on the touch screen. Sit back, stay still and breathe normally while I gather your information. This should take about 30 seconds and then you can tell me how I can help you today. (data collected though biometric sensors)
Avatar: I've gathered your information. Now tell me, what brings you in today?
User: (via auditory sensor) I'm feeling tired, I have a scratchy throat, itchy sinuses and I'm starting to feel aches in my shoulder.
Avatar: Can you point to the area in your shoulders that aches.
User: (touches back and shoulders) (data transmitted via optical sensor)
Avatar: I've also detected a slight fever and elevated blood pressure (data collected through biometric sensors). Have you had a flu shot?
User: No.
Avatar: If we look at this map, we see that the flu is on the rise in your neighborhood, while the common cold is decreasing. This is true at your children's school, as well. Also, an increasing number of your co-workers have gone home with the flu. I'm advising go home, drink fluids, rest and contact your doctor if your temperature rises above 103 degrees. Will that be acceptable to you?
User: Yes.
Avatar: I will send a summary of our session to your email address and your primary doctor. I will also update your personal health record. Shall I contact your supervisor to say you are heading home due to illness?
User: Yes, please [alternatively user keys in “Yes” through user interface]
Avatar: Is there anything else I can do for you?
User: Nope.
Avatar: Ok. I hope you feel better and thank you for staying on top of your health. Goodbye.

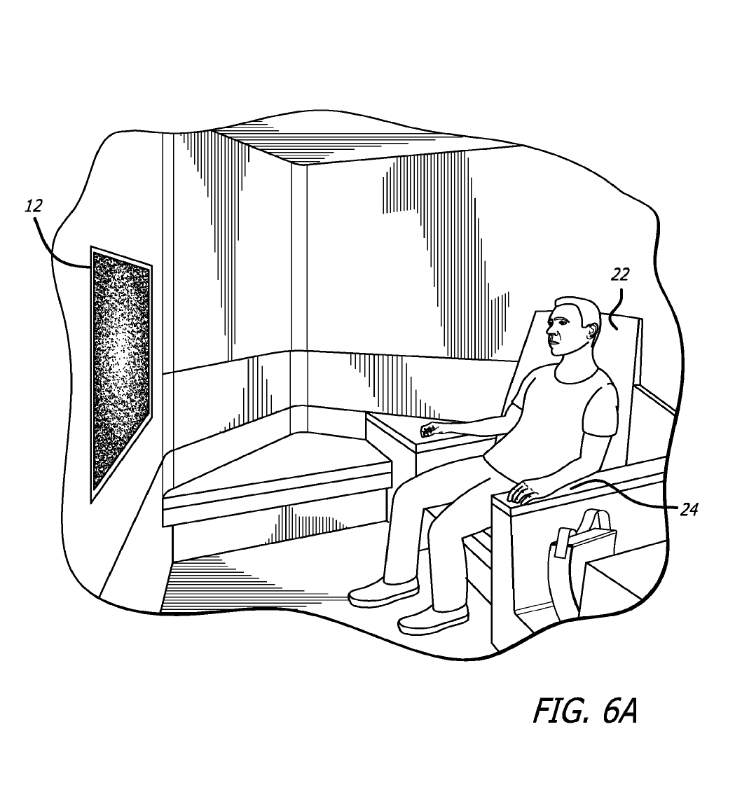

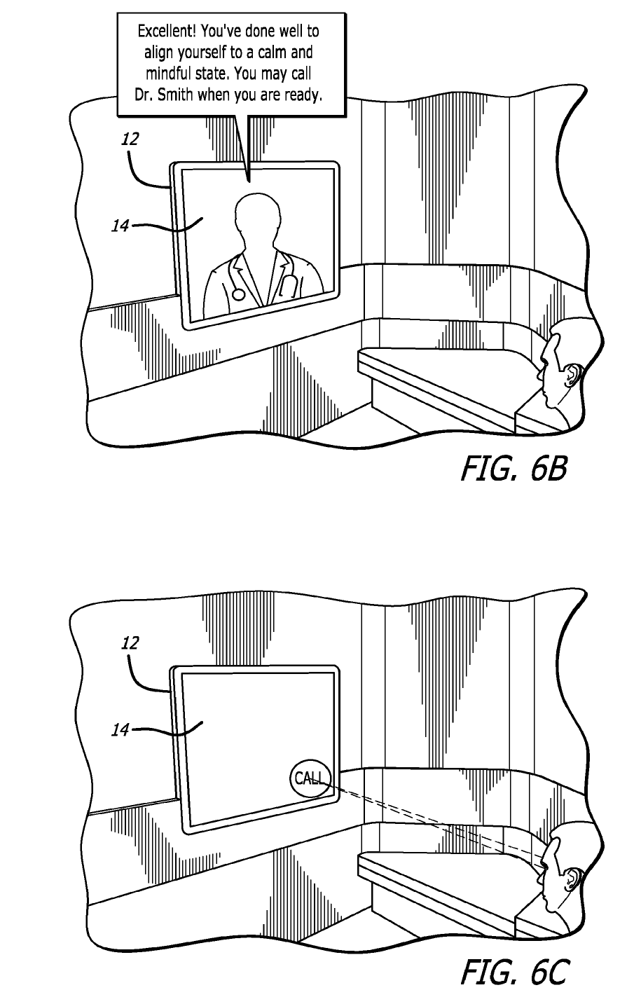

As shown in FIGS. 6A-6C, in a directed interaction, the interaction consists of an interaction between a user and a remote participant. The interaction can take place either directly through the interface 12. Alternatively, an avatar displayed on the user interface 12 may communicate audible and visual prompts and responses received from the remote participant. An exemplary diagnostic exchange in a directed, social-sensing distributed health interaction between a user and an avatar is set forth below:

Avatar: Welcome. Relax and have a seat. You can communicate with me at any time by voice or by making selections on the touch screen. Please select your seating comfort. (chair adjusts based on person's presets, e.g., position and temperature).

Avatar: Before we start your session, Dr. Smith has prescribed a brief stress reducing treatment for you. Please select from the packages offered on the screen, relax and enjoy. I'll return in several minutes when you are ready. (user selects option through I/O).

(data collected though biometric sensors).

(environmental changes ensue responsive to data and inputs)
halo light lowers to 35%; shroud light turns pulsing blue
bladder in back portion of user support inflates
display emits small blue pulse in sync with user breathing

Avatar: Excellent! You've done well to align yourself to a calm and mindful state. You may call Dr. Smith when you are ready.

(user selects call button)

Dr. Smith appears on screen and consultation begins.

Users face becomes agitated (detected by optical sensors and biometric sensor)

(environmental response)
lighting adjusts
cool breeze blows across face

The method of the present invention is a client-server interactive method for providing interactive and adaptive response to biometric and user inputs, and is employed via the kiosk 10 system. The method is implemented by a non-transitory computer readable medium that includes machine-readable instructions for causing a processor to execute the method.

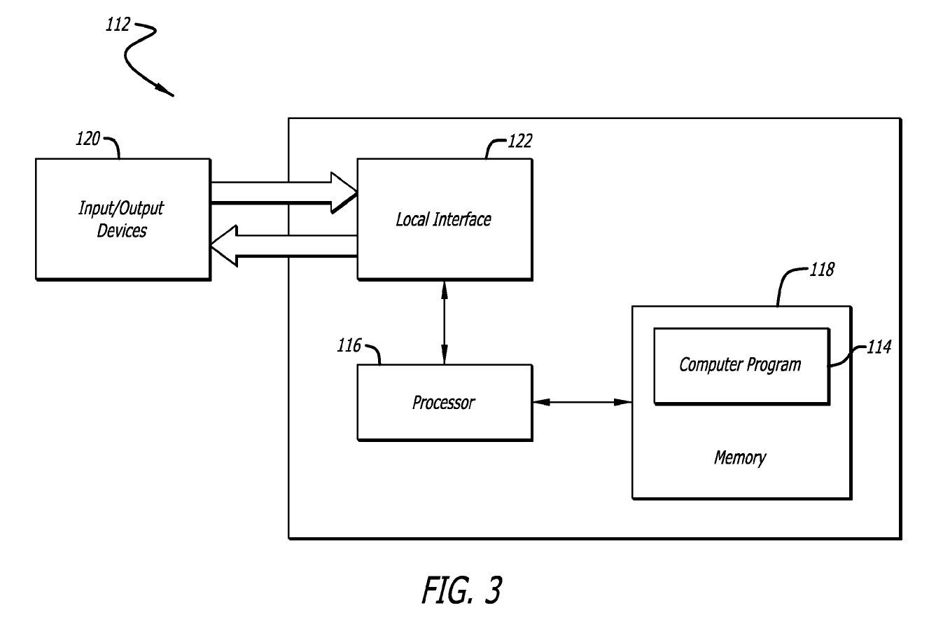

The blocks in FIGS. 2 and 3 can be understood as representing modules, segments, or portions of code which include one or more executable instructions for implementing specific logical functions or steps in the process. Alternate implementations are included within the scope of the embodiments of the present invention in which functions may be executed out of order from that shown or discussed, including substantially concurrently or in reverse order, depending on the functionality involved, as would be understood by those having ordinary skill in the art.

FIG. 3 is a block diagram of a computer or server 112. For purposes of understanding the hardware as described herein, the terms “computer” and “server” have identical meanings and are interchangeably used. Computer 112 includes control system 114. The control system 114 of the invention can be implemented in software (e.g., firmware), hardware, or a combination thereof. In the currently contemplated best mode, the control system 114 is implemented in software, as an executable program, and is executed by one or more special or general purpose digital computer(s), such as a personal computer (PC; IBM-compatible, Apple-compatible, or otherwise), personal digital assistant, workstation, minicomputer, or mainframe computer. An example of a general purpose computer that can implement the control system 114 of the present invention is shown in FIG. 3. The control system 114 may reside in, or have portions residing in, any computer such as, but not limited to, a general purpose personal computer. Therefore, computer 112 of FIG. 3 may be representative of any computer in which the control system 114 resides or partially resides.

Generally, in terms of hardware architecture, as shown in FIG. 3, the computer 112 includes a processor 16, memory 18, and one or more input and/or output (I/O) devices 120 (or peripherals) that are communicatively coupled via a local interface 122. The local interface 122 can be, for example, but not limited to, one or more buses or other wired or wireless connections, as is known in the art. The local interface 122 may have additional elements, which are omitted for simplicity, such as controllers, buffers (caches), drivers, repeaters, and receivers, to enable communications. Further, the local interface may include address, control, and/or data connections to enable appropriate communications among the other computer components.

The processor 116 is a hardware device for executing software, particularly software stored in memory 118. The processor 116 can be any custom made or commercially available processor, a central processing unit (CPU), an auxiliary processor among several processors associated with the computer 112, a semiconductor based microprocessor (in the form of a microchip or chip set), a macroprocessor, or generally any device for executing software instructions. Examples of suitable commercially available microprocessors are as follows: a PA-RISC series microprocessor from Hewlett-Packard Company, an 80×8 or Pentium series microprocessor from Intel Corporation, a PowerPC microprocessor from IBM, a Sparc microprocessor from Sun Microsystems, Inc., or a 8xxx series microprocessor from Motorola Corporation. However, it will be understood that the microprocessor used in the present invention need not be limited to the present commercially available processors.

The memory 118 can include any one or a combination of volatile memory elements (e.g., random access memory (RAM, such as DRAM, SRAM, SDRAM, etc.)) and nonvolatile memory elements (e.g., ROM, hard drive, tape, CDROM, etc.). Moreover, memory 18 may incorporate electronic, magnetic, optical, and/or other types of storage media. The memory 118 can have a distributed architecture where various components are situated remote from one another, but can be accessed by the processor 116.

The software in memory 118 may include one or more separate programs, each of which comprises an ordered listing of executable instructions for implementing logical functions. In the example of FIG. 3, the software in the memory 118 includes the control system 114 in accordance with the present invention and a suitable operating system (O/S) 124.

The control system 114 may be a source program, executable program (object code), script, or any other entity comprising a set of instructions to be performed. When a source program, the program needs to be translated via a compiler, assembler, interpreter, or the like, which may or may not be included within the memory 118, so as to operate properly in connection with the O/S 124. Furthermore, the control system 114 can be written as (a) an object oriented programming language, which has classes of data and methods, or (b) a procedure programming language, which has routines, subroutines, and/or functions, for example but not limited to, C, C++, Pascal, Basic, Fortran, Cobol, Perl, Java, and Ada. In one embodiment, the control system 114 is written in C++. The I/O devices 120 may include input devices, for example but not limited to, a keyboard, mouse, scanner, microphone, touch screens, interfaces for various medical devices, bar code readers, stylus, laser readers, radio-frequency device readers, etc. Furthermore, the I/O devices 120 may also include output devices, for example but not limited to, a printer, bar code printers, displays, etc. Finally, the I/O devices 120 may further include devices that communicate both inputs and outputs, for instance but not limited to, a modulator/demodulator (modem; for accessing another device, system, or network), a radio frequency (RF) or other transceiver, a telephonic interface, a bridge, a router, etc.

When the computer 112 is in operation, the processor 116 is configured to execute software stored within the memory 118, to communicate data to and from the memory 118, and to generally control operations of the computer 112 pursuant to the software. The control system 114 and the O/S 124, in whole or in part, but typically the latter, are read by the processor 116, perhaps buffered within the processor 16, and then executed.

When the control system 114 is implemented in software, as is shown in FIG. 3, it should be noted that the control system 114 can be stored on any non-transitory computer readable medium for use by or in connection with any computer related system or method. In the context of this document, a “non-transitory computer-readable medium” does not reference a signal itself. Instead, a non-transitory computer-readable memory can be any physical means suitable to store, communicate, propagate, or transport the program for use by or in connection with the instruction execution system, apparatus, or device. The computer readable medium can be for example, but not limited to, an electronic, magnetic, optical, electromagnetic, infrared, or semiconductor system, apparatus, device, or propagation medium. The control system 114 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

In another embodiment, where the control system 114 is implemented in hardware, the control system 14 can be implemented with any or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

According to one embodiment, a code segment receives a transmission of a unique identifier that corresponds to one of either a single user or a predetermined class of users. A code segment communicates with an electronic health record database associated with the unique identifier. A code segment receives at least one user health output data measured by at least one biometric sensor within a participative health kiosk. A code segment generates a diagnostic output, and a code segment transmits one of a plurality of command signals that are based on the generated diagnostic output. A codes segment transmits at least one prompt to a user interface and a code segment receives at least one input from a user interface to execute a diagnostic routine.

In one embodiment, a code segment is provided to compare user health output data 28 to at least one of a plurality of user conditions stored in the electronic health record data database. Accordingly, the generated diagnostic output is based on a comparison of the user health output data to the user conditions stored in the health record data database. The generated diagnostic output 30 also may be based on comparing non-user epidemiological data to at least one user health output data. The non-user epidemiological data can be stored in an epidemiological database. A code segment is provided to receive the non-user epidemiological data from either the Internet or a social media feed.

While the specific embodiments have been illustrated and described, numerous modifications come to mind without significantly departing from the spirit of the invention and the scope of protection is only limited by the scope of the accompanying Claims.

What is claimed is:

1. A method for use in a client-server interactive participative health system for providing interactive and adaptive response to biometric and user inputs, the method comprising:
receiving a transmission of a unique identifier, the unique identifier corresponding to one of either a single user or a predetermined class of users;
communicating with a health record database associated with the unique identifier;
receiving at least one user health output data measured by at least one biometric sensor within a participative health kiosk;
generating a diagnostic output;
generating at least one command signal based on the generated diagnostic output; and,
transmitting the generated at least one command signal.

2. The method of claim 1, further comprising the step of electronically communicating with an electronic health records database.

3. The method of claim 1, wherein the generated diagnostic output is based on a comparison of at least one of the user health output data input transmission and an input transmission from the user interface to at least one of a plurality of user conditions stored in the health data database.

4. The method of claim 1, wherein the generated diagnostic output is based on a comparison of at least one of a plurality of non-user epidemiological data to at least one of the user health output data input transmission and an input transmission from the user interface.

5. The method of claim 4, wherein the at least one of a plurality of the non-user epidemiological data is stored in an epidemiological database.

6. The method of claim 4, wherein the at least one of a plurality of the non-user epidemiological data is one of either transmitted and retrieved from the Internet.

7. The method of claim 1, further comprising the step of executing a diagnostic routine by transmitting at least one prompt to a user interface and receiving at least one input from a user interface.

8. The method of claim 1, wherein the at least one of a plurality of command signals each comprises an environmental command signal for changing an environmental setting of an enclosed environment.

9. A method for use in a client-server interactive participative health kiosk for providing interactive and adaptive response to biometric and user inputs, wherein the health kiosk includes a user interface including a display and at least one audio speaker, a user support positioned to provide a user with a view of the user interface, at least one biometric sensor configured to measure real-time biometric data associated with the user, an auditory sensor for receiving vocal input commands from the user, an optical sensor for detecting movement of the user within the kiosk, and a control system configured to communicate with the user interface, the biometric sensors, the auditory sensor, and the optical sensor, the method comprising:
receiving a transmission of a unique identifier, the unique identifier corresponding to one of either a single user or a predetermined class of users;
communicating with an electronic health record database associated with the unique identifier;
receiving at least one user health output data measured from at least one of the biometric, auditory and optical sensors within a participative health kiosk;
generating a diagnostic output based on a comparison of a plurality of non-user epidemiological data to both the user health output data input transmission and an input transmission from the user interface;
transmitting one of a plurality of command signals, the command signal being based on the generated diagnostic output;
transmitting at least one diagnostic routine to the user interface display in response to the diagnostic output, the diagnostic routine being a series of queries and prompts communicated in the form of a digitally generated avatar displayed through the user interface display;
transmitting an alert message to a remote participant based on the health output data, the alert message indicating the attendance status of a user; and
transmitting a display communication to the user display interface, notifying user that an alert message has been transmitted to a remote participant.

10. The method of claim 9, wherein the remote participant is at least one of a non-user human participant and a computer.

11. A non-transitory computer readable medium causing a processor to execute machine-readable instructions to:
receive a transmission of a unique identifier, the unique identifier corresponding to one of either a single user or a predetermined class of users;
communicate with an electronic health record database associated with the unique identifier;
receive at least one user health output data measured by at least one biometric sensor within a participative health kiosk;
generate a diagnostic output; and
transmit one of a plurality of command signals, the command signal being based on the generated diagnostic output.

12. The non-transitory computer readable medium of claim 11, wherein the non-transitory computer readable medium further causes the processor to compare the at least one user health output data to at least one of a plurality of user conditions stored in the electronic health record data database.

13. The non-transitory computer readable medium of claim 12, wherein the generated diagnostic output is based on the comparison of the at least one user health output data to the at least one of a plurality of user conditions stored in the health record data database.

14. The non-transitory computer readable medium of claim 12, wherein the non-transitory computer readable medium further causes the processor to compare at least one of a plurality of non-user epidemiological data to at least one user health output data.

15. The non-transitory computer readable medium of claim 14, wherein the generated diagnostic output is based on the comparison of at least one of the plurality of non-user epidemiological data to the at least one user health output data.

16. The non-transitory computer readable medium of claim 14, wherein the at least one of a plurality of the non-user epidemiological data is stored in an epidemiological database.

17. The non-transitory computer readable medium of claim 12, wherein the non-transitory computer readable medium further causes the processor to receive an input transmission of the least one of a plurality of the non-user epidemiological data from at least one of either the Internet and a social media feed.

18. The non-transitory computer readable medium of claim 11, wherein the non-transitory computer readable medium further causes the processor to transmit at least one prompt to a user interface and receiving at least one input from a user interface to execute a diagnostic routine.

19. The non-transitory computer readable medium of claim 11, wherein the at least one of a plurality of command signals each comprises an environmental command signal for changing an environmental setting of an enclosed environment.
